(19) United States
(12) Patent Application Publication
(10) Pub. No.: US 2026/0218114 A1
(43) Pub. Date: Jul. 30, 2026

(54) METHODS AND SYSTEMS FOR HARVESTING A BIOMASS CONTAINING ALGAL CELLS

(71) Applicant: Neste Oyj, Espoo (FI)

(72) Inventors: Jeffrey Scott KANEL, Porvoo (FI); Cecil Calvert CHURN, III, Porvoo (FI); Cecilia Alexandra STILL, Turku (FI)

(73) Assignee: Neste Oyj, Espoo (FI)

(21) Appl. No.: 19/144,199

(22) PCT Filed: Dec. 22, 2023
(86) PCT No.: PCT/US2023/085723

§ 371 Date: Jun. 27, 2025

Related U.S. Application Data
(60) Provisional application No. 63/477,837, filed on Dec. 30, 2022.

(51) Int. Cl. C12N 1/12; C12M 1/00; C12M 1/26; C12M 1/34; C12N 1/02; C12R 1/89

(52) U.S. Cl. CPC C12N 1/12; C12M 21/02; C12M 23/18; C12M 33/00; C12M 41/06; C12M 41/32; C12M 41/34; C12N 1/02; C12R 2001/89

(57) ABSTRACT

The present disclosure relates to methods and systems for harvesting a biomass containing algal cells. The methods include obtaining a stream containing an algal growth medium, algal cells and/or prokaryotes from an algal aquaculture, treating the stream by reducing or excluding light to obtain an algal harvester feed stream having a dissolved oxygen content less than about 8 mg dissolved oxygen (DO)/liter, and feeding at least part of the algal harvester feed stream into a harvester. Systems for providing the above methods, an algal harvester feed stream and compositions including algal cells are also provided.

### FIELD

The present disclosure relates to methods and systems for harvesting a biomass containing algal cells from streams and/or agal aquaculture systems, especially those that use algae, bacteria, archaea, or any combination thereof to produce valuable biological products. The present disclosure also provides uses of an algal harvester feed stream containing algae or products thereof, including algal oil, proteins, and carotenoids. Systems for performing these methods, compositions containing the products produced, and the products obtained by the methods disclosed herein are also provided.

### BACKGROUND INFORMATION

Algal biomass, or biomass, has been recognized as a potential source of valuable products, such as oil for renewable fuels and sustainable chemicals, including proteins and carotenoids. Microalgae are a unique subset of algae that can be grown in bioreactors under controlled conditions. Once the microalgae are grown, they must be efficiently harvested without significant loss or degradation. One significant source of degradation is oxidation of the valuable products. During a harvesting method, valuable components of algae can be exposed to oxygen, which results in the degradation and loss of e.g., algal oil and carotenoids. An important source of oxygen in the harvesting method is that which is dissolved and entrained in the algal growth medium being fed to the harvester.

Since algae are like plants, they take in carbon dioxide, and release oxygen to a growth medium during the photosynthetic daylight hours. Even though lower salinity bacteria, halo-tolerant bacteria and archaea that can cohabitate the algal growth medium, take in oxygen and release carbon dioxide, the dissolved oxygen content of the algal growth medium can increase essentially during the daylight hours. Thus, high contents of dissolved oxygen are introduced into the harvester causing degradation of the algal oil and carotenoids.

One way to reduce the potential for oxidative degradation to occur in the harvester is using an inert gas as the flotation gas, such as nitrogen, carbon dioxide, and the noble gases, in an adsorptive bubble separation method. However, the storage and use of an inert gas is expensive, so there is a need to develop a lower-cost solution to protect the algal oil and carotenoids during the adsorptive bubble separation process. We have surprisingly found a way to reduce the oxygen content in the feed to the harvester that can significantly reduce the amount of loss and degradation of algal oil and carotenoids in the harvester. For example, the need for an inert gas to reduce the amount of loss and degradation of algal oil and carotenoids in the adsorptive bubble separation process can be reduced with the methods and system of the present disclosure.

Harvesting approaches are generally described in Biomass Program, National Algal Biofuels Technology Roadmap, 2010, U.S. Department of Energy, Energy Efficiency & Renewable Energy. Flocculation-flotation harvesting mechanism of Dunaliella salina: from nanoscale interpretation to industrial optimization is described in Besson et al., Water Research, 2019, vol. 155, p 352-361. There are a number of harvesting methods that have been disclosed for harvesting algae, and they include, but are not limited to, centrifugation, precipitation, sedimentation, adsorptive bubble processes, and combinations thereof. Some of these harvesting methods have also been disclosed in U.S. Pat. No. 5,951,875.

There are four known methods that can be used to reduce the dissolved oxygen content in the feed to an algal harvester unit. The first known method to reduce the oxygen content in a harvester feed stream relies on sparging the harvester feed stream with an inert gas. However, this method requires special equipment that consumes both capital and operating expenses.

A second known method of reducing oxidation is to add a suitable antioxidant to the harvester system. Since the algal growth medium is aqueous and water-based, water-soluble antioxidants are preferred, such as ascorbic acid, propionic acid, carnosic acid, other compounds, and combinations thereof. This method introduces a chemical additive to the harvesting process, which can require additional modifications and may not be acceptable at all in some locations. Due to environmental and cost reasons, added chemicals should be avoided or at least their amount should be reduced in harvesting methods and systems.

A third known method is to use an inert gas as the flotation gas in an adsorptive bubble separation method. This method has been disclosed in U.S. Pat. No. 5,951,875.

A fourth known method is to use sodium sulfite to deoxygenate the feed with a cobalt catalyst such as cobalt chloride. In such a method, the cobalt chloride dose is typically no more than 0.05 mg/L. The sodium sulfite reacts with the oxygen as follows: Na₂SO₃+0.5 O₂=Na₂SO₄. This stoichiometry shows that theoretically 7.875 mg/L of sodium sulfite will react with each mg/L of oxygen present at the time of dosing. However, it is common to add 1.5 times the required amount (i.e., 11.8 mg/mg O₂) to ensure complete deoxygenation. As above, this method also introduces a chemical additive to the harvesting process, which can require additional modifications and may not be acceptable at all in some locations. For environmental and cost reasons, added chemicals should be avoided or at least their amount should be reduced in harvesting methods and systems.

Despite the known approaches described above, there exists a need for a simple, environmentally-friendly and effective method to recover an algal biomass on a large scale, whilst reducing the amount of products lost to oxidation and degradation.

To address the foregoing need, the inventors of the present disclosure provide efficient and low-cost methods and systems for reducing the amount of oxygen that is carried into the harvester as dissolved oxygen, in the algal growth medium.

### SUMMARY

Disclosed herein is a method for harvesting a biomass containing algal cells, the method including (i.e., comprising) at least one or more of the following: obtaining a stream containing an algal growth medium, algal cells and/or prokaryotes from an algal aquaculture; treating the stream by reducing or excluding the stream's exposure to light to obtain an algal harvester feed stream having a dissolved oxygen content less than about 8 mg dissolved oxygen (DO)/liter; and/or feeding at least a part of the algal harvester feed stream into a harvester.

Disclosed herein is also a use of an algal harvester feed stream containing a dissolved oxygen content of less than about 8 mg dissolved oxygen (DO)/liter for extracting algae and/or products including algal oil, proteins and carotenoids.

Disclosed herein is a system for reducing degradation and/or loss of algal products by oxidation during harvesting of a biomass containing algal cells, the system including at least one or more of the following: a covered canal configured to reduce or eliminate light exposure of a stream containing an algal growth medium, algal cells and/or prokaryotes; and/or a harvester configured to communicate with the covered canal.

Uses of the systems to reducing degradation, optionally degradation by oxidation, and/or loss of algal cells or products thereof during harvesting of a biomass containing algal cells are also provided herein.

Disclosed herein is an algal harvester feed stream composition that includes at least one or more of the following: algae or products thereof, algal growth medium, and/or a dissolved oxygen content of less than about 8 mg dissolved oxygen (DO)/liter.

Also disclosed herein is an algal biomass obtainable by any method disclosed herein, wherein algal oil, proteins and/or carotenoids are recoverable from the algal biomass.

### BRIEF DESCRIPTION OF THE DRAWINGS

Other features and advantages of the present disclosure will be apparent to those skilled in the art reading the following detailed description in conjugation with the exemplary embodiments illustrated in the drawings, wherein:

FIG. 1 shows an exemplary system for controlling dissolved oxygen content (140) in an algal harvester feed stream (150) entering an algal harvester (180) by passing it through a covered canal (130) having a specific design and/or structure that facilitates control of the dissolved oxygen content.

FIG. 2 shows an exemplary dissolved oxygen content cycle of an algal culture containing Dunaliella salina and halotolerant bacteria, as a function of time, in the presence and absence of solar radiation.

FIG. 3 shows an exemplary dissolved oxygen uptake rate in an algal culture containing Dunaliella salina and halotolerant bacteria, as a function of time, in the absence of solar radiation.

FIG. 4 illustrates an exemplary dissolved oxygen uptake rate in an algal culture containing Dunaliella salina and halotolerant bacteria, as a function of time, in the absence of solar radiation.

### DETAILED DESCRIPTION

One aspect of the present disclosure is a method for harvesting a biomass containing algal cells, the method including at least one or more of the following: obtaining a stream containing an algal growth medium, algal cells and/or prokaryotes from an algal aquaculture; treating the stream by reducing or excluding light to obtain an algal harvester feed stream having a dissolved oxygen content less than about 8 mg dissolved oxygen (DO)/liter; and/or feeding at least a part of the algal harvester feed stream into a harvester.

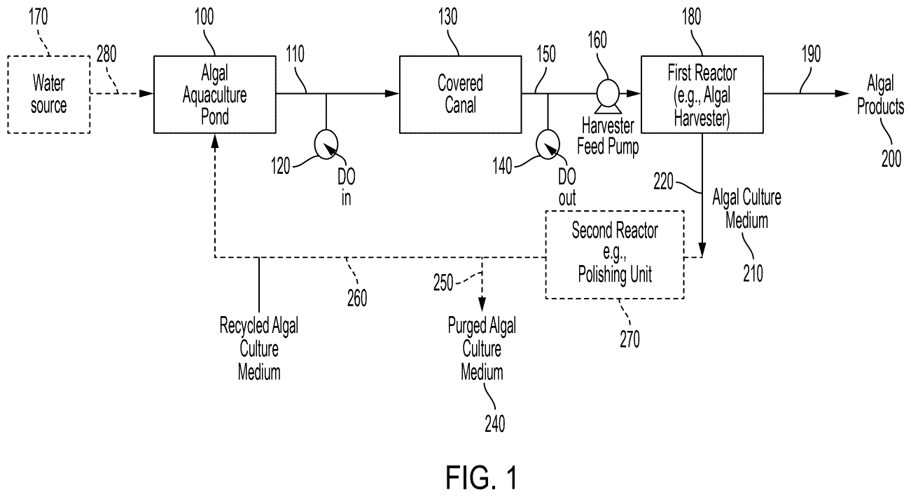

An exemplary embodiment of a method for harvesting a biomass containing algal cells is depicted in FIG. 1. In FIG. 1, a stream containing an algal growth medium, algal cells and/or prokaryotes is obtained from an algal aquaculture pond (100). The algal aquaculture pond (100) can optionally be supplied with an aqueous medium from a water source (170) through a transport line (280). The stream containing algal growth medium, algal cells and/or prokaryotes possesses an initial dissolved oxygen content (denoted “DO-in”, 120). The stream is transferred to a covered canal (130) through a transport line (110). In the covered canal (130), the stream is treated with reduced light exposure to obtain an algal harvester feed stream (150) having a dissolved oxygen content less than about 8 mg dissolved oxygen (denoted “DO-out”, 140). The algal harvester feed stream (150) is fed into a first reactor (e.g., an algal harvester, 180) through a harvester feed pump (160). In the algal harvester (180), algal products (200) and algal culture medium (210) are separated from the algal harvester feed stream (150). The algal products (200) are removed from the algal harvester (180) via a recovery line (190) and transferred to further downstream processes.

The algal culture medium (210) can optionally be transported through a recovery line (220) to a second reactor (e.g., a polishing unit, 270) containing microorganisms capable of metabolizing any amount of glycerol present in the algal culture medium (210). The algal culture medium (210) then leaves the polishing unit (270) through a recovery line (230). At least a portion of the algal culture medium (210) leaving the polishing unit (270) can be recovered through a transport line (250) as a purged algal culture medium (240). The remaining portion of the algal culture medium (210) is recycled back to the algal aquaculture pond (100) through a transport line (260).

In exemplary embodiments, the algal harvester feed stream has a dissolved oxygen content of less than about 7, less than about 6, less than about 5, less than about 4, less than about 3, less than about 2, or less than about 1 mg dissolved oxygen (DO)/liter.

The term “one or more” used herein is interchangeable with “at least one”.

The algal aquaculture pond can be or include a pond open to the atmosphere (e.g., a mechanically agitated raceway pond design, a pond without mechanical agitation, a multistep aquaculture including multiple algal aquaculture ponds), a pond closed to the atmosphere (e.g., a greenhouse-covered pond), and optionally contain a closed or semi-closed bioreactor (e.g., a fermenter), a bubble column, a tubular bioreactor, or a combination thereof. In an exemplary embodiment, the algal aquaculture pond can be any type of algal aquaculture pond used to grow algae, including, but not limited to open pond, closed pond, raceway pond, enclosed or partly enclosed photobioreactor, tubular reactor, flat panel (i.e., flat-plate) reactor, column reactor, enclosed raceway, covered pond, open raceway pond, earthen pond, pond in a greenhouse, fermenter, naturally occurring body of water, solar salt pond, or any combinations thereof.

In an exemplary embodiment the algal aquaculture pond contains or is an open pond, bioreactor, or a combination thereof.

In exemplary embodiments, the method includes a continuous flow process. For example, the steps of the method are performed in a continuous operation. The products from any of the method steps disclosed herein can be the feed for another cycle or step of the same method.

Additionally, the stream containing algal growth medium, algal cells and/or prokaryotes can either be in direct or indirect communication with a harvester, such as an algal harvester. When the stream containing algal growth medium, algal cells and/or prokaryotes is in indirect communication with a harvester, an intermediate communication with a pipe, a tube or a covered canal can exist. In one exemplary embodiment, the stream flows between the algal aquaculture pond and the pipe, tube or the covered canal. The stream can contain an initial dissolved oxygen content going into the pipe or the covered canal (also denoted “DO-in”) and a different dissolved oxygen content coming out of the pipe or the covered canal (denoted “DO-out”).

As used herein, the term “algal harvester feed stream” refers to the stream leaving the pipe or the covered canal.

In exemplary embodiments, the method includes treating the stream containing algal growth medium, algal cells and/or prokaryotes by reducing and/or excluding light exposure using a pipe, a tube and/or a cover. For example, a covered canal including a pipe, a tube and/or a cover can be used to reduce and/or exclude the stream from light exposure.

In exemplary embodiments, the method includes treating the stream containing algal growth medium, algal cells and/or prokaryotes to reduced or no light exposure by flowing the stream through an opaque pipe or an opaque tube in a covered canal.

In exemplary embodiments, the stream containing algal growth medium, algal cells and/or prokaryotes can be treated by reducing or excluding light by passing the stream through a region that excludes solar radiation during the day. The region that excludes solar radiation can include a pipe, tube and/or a covered canal.

In exemplary embodiments, the method reduces the light exposure of the stream containing algal growth medium, algal cells and/or prokaryotes at least by 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%. Light exposure can be from either sunlight or artificial light. Reducing the light exposure of the stream by the above amount can reduce the dissolved oxygen content of the stream before harvesting.

In exemplary embodiments, the stream containing algal growth medium, algal cells and/or prokaryotes can be treated during the presence of external sunlight or artificial light. The presence of external sunlight or artificial light can be outside of a covered canal.

In exemplary embodiments, the stream containing algal growth medium, algal cells and/or prokaryotes can be treated by reducing or excluding light in a pipe, with a cover over or on the pipe, with a deep canal, or a combination thereof.

The algal aquaculture pond can include one or more micrcroorganisms including autotrophic microbes, such as algae, bacteria, archaea and/or combinations thereof. In an exemplary embodiment, the one or more microorganisms contain eukaryotic algae, prokaryotes (such as one or more bacteria and/or one or more archaea) or combinations thereof.

The microorganisms can also include one or more heterotrophic microbes, one or more halophilic microbes, one or more halotolerant microbes, one or more microalgae, one or more anaerobic microbes, one or more aerobic microbes, or combinations thereof.

In exemplary embodiments, the microorganisms contain eukaryotic algae, prokaryotes, or combinations thereof. Prokaryotes that can be present within the microorganisms can include bacteria and/or archaea. The eukaryotic algae can be used to create a biomass (e.g., an algal biomass).

In exemplary embodiments, the microorganisms can include one or more algae from divisions Bacillariophyta, Chlorophycophyta, Chrysophycophyta, Cyanophycophyta, Cryptophycophyta, Phaeophycophyta, Pyrrhophycophyta, Rhodophycophyta and combinations thereof are suitable for use in the present disclosure.

In exemplary embodiments, the microorganisms can include one or more algae from the divisions of Chlorophycophyta, Phaeophycophyta, Chrysophycophyta, Cyanophycophyta, Cryptophycophyta, Pyrrhophycophyta and/or Rhodophycophyta, which are adaptable to saline water as a growth medium; or one or more microalgae species selected from, but not limited to, Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella kone, Dunaliella tertiolecta, Dunaliella viridis, Euglena gracilis, Fragilaria sp., Fragilaria sublinearis, Gracilaria sp., Haematococcus pluvialis, Hantzschia sp., Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea Nostoc commune, Nostoc flagellaforme, Pavlova gyrens, Peridinium sp., Phaeodactylum tricornutum, Pleurochrysis carterae, Porphyra sp., Porphyridium aerugineum, Porphyridium cruentum, Prymnesium sp., Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema sp., Skeletonema costatum, Spirogyra sp., Schizochytrium limacinum, Stichococcus bacillaris, Synechococcus sp., Tetraselmis sp., Tolypothrix sp., and genetically-engineered varieties or combinations (mixtures, or mixed cultures) of these microalgal species.

In exemplary embodiments, the one or more algae or microalgae present in the microorganisms are selected from one or more species from the following genera: Acutodesmus, Achnahtes, Amphipora, Amphora, Anabaena, Ankistrodesmus, Arthrospira (also known as Spirulina), Asteromonas, Asterionella, Boekelovia, Borodinella, Botryococcus, Bracteacoccus, Carteria, Chaetoceros, Chlamydomonas, Chlorella, Chlorococcum, Chlorogonium, Chloromonas, Chroomonas, Chrysophaera, Ceratium, Closterium, Coccolithus, Coelastrella, Coscinodiscus, Cosmarium, Cricosphaera, Crocosphaera, Crypthecodinium, Cryptomonas, Cyanocystis, Cyanospira, Cyclotella, Desmodesmus, Ditylum, Dunaliella, Ellipsoidon, Emiliania, Eremosphaera, Euglena, Fragilaria, Franceia, Galdieria, Gracilaria, Graesiella, Guinardia, Haematococcus, Halocafeteria, Halospirulina, Hantzschia, Hymenomonas, Isochrysis, Lepocinclis, Limnothrix, Micractinium, Microactinium, Microcystis, Monochrysis, Monodus, Monoraphidium, Muriellopsis, Nannochloris, Nannochloropsis, Navicula, Neochloris, Neospongiococcum, Nephrochloris, Nephroselmis, Nitzschia, Nodularia, Nostoc, Ochromonas, Oedogonium, Oocystis, Oscillatoria, Ostreococcus, Parachlorella, Pavlova, Peridinium, Phaeodactylum, Picochlorum, Platymonas, Pleurochrysis, Pleurococcus, Porphyra, Porphyridium, Prochlorococcus, Prototheca, Prymnesium, Pseudanabaena, Pseudochlorella, Pseudochoricystis, Pseudoneochloris, Pyramimonas, Pyrobotrys, Rhodomonas, Scenedesmus, Schizochytrium, Scytonema, Skeletonema, Spirogyra, Stichococcus, Synechococcus, Tetrachlorella, Tetradesmus, Tetraselmis, Thalassiosira, Tisochrysis, Tolypothrix, Tribonema, Trichodesmium, Ulothrix, Vaucheria, Viridiella, Volvox, and genetically-engineered varieties or combinations (mixtures, mixed cultures, co-cultures or synthetic co-cultures) thereof.

In exemplary embodiments, the one or more algae or microalgae present in the microorganisms are selected from the group including or consisting of Dunaliella sp., Dunaliella bardawil, Dunaliella kone, Dunaliella salina, Dunaliella bioculata, Dunaliella granulata, Dunaliella maritima, Dunaliella minuta, Dunaliella parva, Dunaliella peircei, Dunaliella polymorpha, Dunaliella primolecta, Dunaliella pseudosalina, Dunaliella quartolecta, Dunaliella terricola, Dunaliella tertiolecta, and Dunaliella viridis.

In exemplary embodiments, the one or more algae or microalgae present in the microorganisms are selected from the group including Dunaliella sp., Dunaliella bardawil, Dunaliella salina, Dunaliella kone, Dunaliella tertiolecta, Dunaliella parva and Dunaliella viridis, and any combination thereof.

In exemplary embodiments, the algae or microalgae can be Dunaliella sp., Dunaliella salina, and/or Dunaliella bardawil, or a combination thereof.

The algae or microalgae which can be present in the microorganisms can include one or more microalgal species (including diatoms, coccolithophorids and dinoflagellates) selected from, but not limited to, Amphora sp., Ankistrodesmus sp., Arthrospira (Spirulina) plantesis, Botryococcus braunii, Chlamydomonas sp., Chlamydomonas reinhardtii, Chlorella protothecoides, Chlorella sp., Closterium sp., Cosmarium sp., Crypthecoddinium cohnii, Cyclotella sp., Dunaliella salina, Dunaliella kone, Dunaliella bardawil, Dunaliella tertiolecta, Haematococcus pluvialis, Hantzschia sp., Nannochloris sp., Nannochloropsis sp., Navicula sp., Neochloris oleoabundans, Nitzschia sp., Phaeodactylum tricornutum, Scenedesmus sp., Schizochytrium limacinum, Stichococcus sp., Tetraselmis suecica, and Thalassiosira pseudonana, and genetically-engineered varieties or combinations (mixtures, or mixed cultures) of these microalgal species.

The algae or microalgae which can be present in the microorganisms can also include algae with flagella, cilia and/or eyespots. Flagella are a tail-like projection that protrude from the cell body of certain algae and function in locomotion. Cilia are an adaptation that allows independent cellular creatures, like algae, to move around in search of food. Photosensitive eyespots are found in some free-swimming unicellular algae. Photosensitive eyespots are sensitive to light. They enable the algae to move in relation to a light source. Such algae have the capability of independent motion, phototaxis, and can move towards the surface during daylight. Phototaxis is the movement of microalgae in response to light. For example, certain algae (e.g., Dunaliella) can perceive light by means of a sensitive eyespot and move to regions of higher light concentration to enhance photosynthesis.

The algae or microalgae which can be present in the microorganisms also include marine algae that thrive at salt concentrations above that found in seawater. Suitable marine algae can be selected from, but are not limited to, Amphora sp. (diatom), Arthrospira sp., Arthrospira (Spirulina) obliquus, Arthrospira (Spirulina) platensis, Chlorella sp., Chlorella fusca, Chlorella protothecoides, Chlorella pyrenoidosa, Chlorella stigmataphora, Chlorella vulgaris, Chlorella zofingiensis, Dunaliella sp., Dunaliella bardawil, Dunaliella salina, Dunaliella kone, Dunaliella tertiolecta, Dunaliella viridis, Isochrysis galbana, Microcystis sp., Nannochloropsis sp., Nannochloropsis salina, Navicula sp. (diatom), Navicula saprophila (diatom), Nitzschia laevis (diatom), Nitzschia alba (diatom), Nitzschia communis (diatom), Nitzschia paleacea (diatom), Nitzschia closterium (diatom), Nitzschia palea, (diatom), and genetically-engineered varieties or combinations (mixtures, or mixed cultures) of these algal species.

In exemplary embodiments, the algae in the microorganisms is or includes microalgae. In other exemplary embodiments, the algae or microalgae have not been genetically modified or do not originate from genetically engineered algae or microalgae.

The microorganisms can originate from at least one or a combination of plant, algae, microorganism, bacteria, or microalgae feedstock sources. Suitable algae or microalgae feedstock sources can be derived from reactors that include, but are not limited to, tubular reactors, photobioreactors, enclosed raceways, covered ponds, open raceways, open ponds, earthen ponds, ponds in greenhouses, clear plastic bags hung either indoors or outdoors, fermenters, naturally occurring bodies of water, the sea, the ocean, solar salt ponds, and combinations thereof.

The stream containing algal growth medium, algal cells and/or prokaryotes can also contain a biomass. The biomass can contain natural products formed from microorganisms.

The expression “natural products” refers to products which are naturally produced or found within an environment of a living organism. Natural products can include those which are hydrophobic, hydrophilic, or amphipathic.

In exemplary embodiments, the biomass can contain natural products produced by a plant, a microbe, an algae or microalgae species, these products including lipids, algal lipids, carotenoids, fatty acids, algal fatty acids, triacylglycerols, diacylglycerols, monoacylglycerols, oils, algal oils, chlorophyll, glycerol, phospholipids, carbohydrates, fibers, proteins or combinations thereof.

In exemplary embodiments, the stream containing algal growth medium, algal cells and/or prokaryotes and/or the algal harvester feed stream contains bacteria, archaea, and/or microalgae, optionally having a balanced bacteria: microalgae ratio or a balanced prokaryote: microalgae ratio. The bacteria and/or archaea can be halo-tolerant. The microalgae can also be halo-tolerant. Thus, there can be a balanced halo-tolerant bacteria: halo-tolerant microalgae ratio or a balanced halo-tolerant prokaryote: halo-tolerant microalgae ratio.

In exemplary embodiments, the stream containing algal growth medium, algal cells and/or prokaryotes and/or the algal harvester feed stream contains bacteria and/or microalgae, optionally having a balanced bacteria: microalgae ratio. In exemplary embodiments, the ratio of bacteria: microalgae is above about 1:1, above about 10:1, above about 100:1, above about 1000:1, above about 10,000:1, above about 100,000:1, above about 1,000,000:1, above about 10,000,000:1, e.g. such that the turbidity due to bacterial growth does not negatively impact the algal growth.

In exemplary embodiments, the bacteria and/or the microalgae consume oxygen during respiration and produce carbon dioxide.

In exemplary embodiments, the method includes diminishing an amount of the dissolved oxygen in the stream containing algal growth medium, algal cells and/or prokaryotes by respiration during treating.

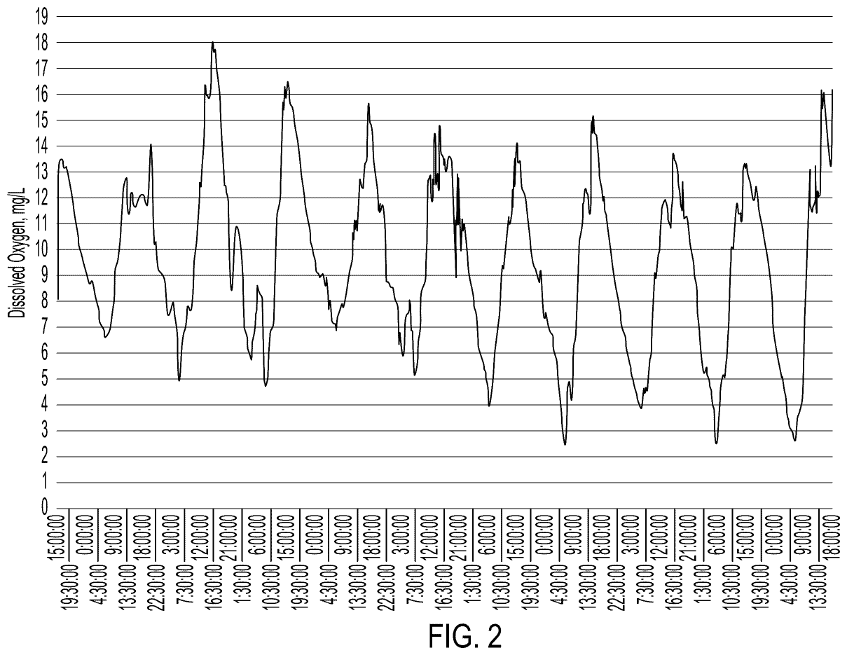

For example, FIG. 2 depicts an exemplary dissolved oxygen content cycle as a function of time of Dunaliella salina, halotolerant bacteria, and archaea, with the measured dissolved oxygen content being reduced at night by the absence of solar radiation. In FIG. 2 the dissolved oxygen (DO) content was measured every 15 minutes with an optical density dissolved oxygen probe supplied by YSI-Xylem. This is an example of how the measured dissolved oxygen content of the stream containing algal growth medium, algal cells and/or prokaryotes would be reduced as it travels through a covered canal.

In exemplary embodiments, the method includes reducing degradation and/or loss of algae and/or products thereof in a harvester; and optionally reducing oxidation degradation of the algal harvester feed stream in the harvester.

The microorganisms can have a residence time in the algal aquaculture pond from 1 day to 50 days, from 2 days to 50 days, from 5 days to 50 days, from 10 days to 50 days, from 20 days to 50 days or any time period falling within these ranges.

In exemplary embodiments, the treating time or mean residence time of the stream containing algal growth medium, algal cells and/or prokaryotes e.g., in a covered canal, pipe or tube can be from about 2 to about 30, from about 5 to about 25, or from about 8 to about 20 hours. For example, the mean residence time can be from about 9 to about 17 hours. In another example, the mean residence time can be from 11 to about 15 hours, such as about 14 hours.

In exemplary embodiments, the method includes controlling the dissolved oxygen content of the algal harvester feed stream by varying the treatment time or mean residence time of the stream containing algal growth medium, algal cells and/or prokaryotes in the covered canal or pipe or tube.

In exemplary embodiments, the dissolved oxygen content in the stream containing algal growth medium, algal cells and/or prokaryotes can be consistently reduced at a rate of between about 0.1 and 5 mg DO/liter/hour, from about 0.2 and 3 mg DO/liter/hour, or from 0.4 and 1.7 mg DO/liter/hour. For example, the dissolved oxygen content in the algal harvester feed stream can be less than about 1, 2, 3, 4, 5, 6, 7, or 8 mg DO/liter.

In exemplary embodiments, the method includes consistently reducing the dissolved oxygen content in the stream containing algal growth medium, algal cells and/or prokaryotes at a rate of between about 0.4 and 1.7 mg DO/liter/hour.

In exemplary embodiments, the dissolved oxygen content in the algal harvester feed stream is less than about 7 mg DO/liter, less than about 5 mg DO/liter, less than about 3 mg DO/liter, and/or less than about 1 mg DO/liter.

In exemplary embodiments, the target or control setpoint of dissolved oxygen in the algal harvester feed stream can be based on the concentration of oxidizable products in the algal harvester feed stream. For example, the target dissolved oxygen content in the algal harvester feed stream can be set to a concentration of some percentage of the concentration of an oxidizable product (e.g., beta-carotene, alpha-carotene, lutein, zeaxanthin, fatty acids) in the algal harvester feed stream. Specifically, the molar ratio of dissolved oxygen to one or more specific oxidizable product(s) can be less than about 1:1, less than about 1:2, less than about 1:4, less than about 1:6, less than about 1:8, less than about 1:10, less than about 1:50, less than about 1:100, less than about 1:500, or less than about 1:1000. In exemplary embodiments, the concentration of dissolved oxygen in the algal harvester feed stream is such so that the molar ratio of dissolved oxygen to beta-carotene is less than about 1:1, less than about 1:1.5, less than about 1:2, less than about 1:4, less than about 1:6, less than about 1:8, less than about 1:10, less than about 1:50, less than about 1:100, or less than about 1:500.

In exemplary embodiments, the dissolved oxygen amount in the stream containing algal growth medium, algal cells and/or prokaryotes and/or the algal harvester feed stream is 0 mg DO/liter, from 0 to about 1 mg DO/liter, or from about 1 to about 8 mg DO/liter.

In exemplary embodiments, the algal growth medium in the algal aquaculture pond or the stream containing algal growth medium, algal cells and/or prokaryotes has an initial measured daily maximum DO-in value of dissolved oxygen concentration greater than about 9 mg DO/liter.

In exemplary embodiments, the dissolved oxygen content in the algal harvester feed stream (DO-out) is less than 60%, less than 55%, less than 50%, less than 45%, less than 40%, less than 35%, less than 30%, less than 25%, less than 20%, less than 15%, less than 10%, or less than 5%, relative to maximum dissolved oxygen content observed in the stream containing algal growth medium, algal cells and/or prokaryotes. In exemplary embodiments, the reduced dissolved oxygen content in the algal harvester feed stream occurs after the stream containing algal growth medium, algal cells and/or prokaryotes has been treated in a covered canal, tube or pipe for at least 24 hours.

In exemplary embodiments, the DO-in content can be reduced by about 45 to 100% when compared to the DO-out content.

In exemplary embodiments, there can be about a 50% drop in the dissolved oxygen content from that measured in the stream containing algal growth medium, algal cells and/or prokaryotes relative to that in the algal harvester feed stream. For example, there can be about a 51%, 52%, 53%, 54%, 55% drop in the dissolved oxygen content.

In exemplary embodiments, the stream containing algal growth medium, algal cells and/or prokaryotes has a dissolved oxygen reduction rate ranging from about 0.49 to about 1.7 mg DO/liter/hour when in a covered canal, tube or pipe.

In exemplary embodiments, the mean residence time of the stream containing algal growth medium, algal cells and/or prokaryotes in the covered canal, tube or pipe can drive the dissolved oxygen content of the algal harvester feed stream exiting the covered canal, tube or pipe to 6.95 mg DO/liter, at a dissolved oxygen reduction rate of 0.49 mg DO/liter/hour. Or, for example a dissolved oxygen reduction rate of 0.45 mg DO/liter/hour.

In exemplary embodiments, the maximum dissolved oxygen content in the stream containing algal growth medium, algal cells and/or prokaryotes, as measured by DO-in, can be from about 10.50 to about 30.50 mg DO/L. In exemplary embodiments, the minimum dissolved oxygen content in the algal harvester feed stream, as measured by DO-out, can be from about 0.25 to about 16.75 mg DO/L.

In exemplary embodiments, the method includes reducing the dissolved oxygen content in the algal harvester feed stream by more than 45%, relative to the maximum dissolved oxygen content observed in stream containing algal growth medium, algal cells and/or prokaryotes in a 24-hour period.

The dissolved oxygen contents disclosed herein can be measured by any means known in the art, including probes that use polarographic, galvanic, and luminescent methods to measure dissolved oxygen contents. Polarographic methods work well at salinities less than seawater. Galvanic methods work well at elevated salt levels, but long-term use can lead to corrosion of the galvanic cell. The LDO or luminescent probes worked well at all salinities and was the method used in the examples disclosed herein.

Dissolved oxygen probes or sensors that measure a current as oxygen is reduced at a cathode are those that involve galvanic means (K. H. Mancy, W. C. Westgarth. J. Wat. Pollut. Control Fed., 34 (1962), pp. 1037-1051) and those that involve polarographic means (D. E. Carritt and J. W. Kanwisher, Anal. Chem. 1959, 31, 1, 5-9). Both can be used in the systems and processes disclosed herein and both use an electrode system where dissolved oxygen reacts at the cathode to produce a current. This current is commonly expressed as the oxidation reduction potential (ORP). ORP is measured in millivolts (mV). Like pH, it is not a measurement of concentration directly, but of activity level. In a solution with a single component (oxygen), ORP indicates concentration. As long as the oxidation-reduction potential (ORP) is maintained at positive Eh values, where Eh is a measure of the redox (oxidation-reduction) state of solutes in a solution, and the aqueous medium remains aerobic. A third general class of dissolved oxygen probes or sensors that can be used in the systems and processes disclosed herein are those that use fluorescence quenching, for example, thus containing metal organic ruthenium complexes. These probes or sensors generally include a sensor tip, optical fibers, an exciting light source, a photomultiplier, and a signal amplifier. The intensity of the fluorescence emitted is a function of the dissolved oxygen concentration. The biggest advantages of fluorescence-based probes or sensors are inherent reliability and low maintenance. Low maintenance and the absence of membranes or electrolyte are key advantages of the fluorescent-based oxygen probes or sensors.

The dissolved oxygen contents measured by these probes can include oxygen that is both dissolved in a thermodynamic sense, as well as oxygen that supersaturates the medium. In this case, the term dissolved oxygen includes both truly dissolved oxygen in a thermodynamic sense, as well as oxygen that supersaturates the medium.

In exemplary embodiments, the method includes monitoring the dissolved oxygen content of the stream containing algal growth medium, algal cells and/or prokaryotes and/or the algal harvester feed stream using an optical density dissolved oxygen probe, or any other method known in the art; and optionally monitoring the dissolved oxygen content when the stream containing algal growth medium, algal cells and/or prokaryotes enters a pipe such as an opaque pipe, a tube such as an opaque tube or a covered canal and/or when the algal harvester feed stream exits the pipe such as the opaque pipe, the tube such as the opaque tube or the covered canal. The dissolved oxygen contents disclosed herein can be based on measurements made with an optical density dissolved oxygen probe.

In exemplary embodiments, the mean dissolved oxygen content is the mean of measured dissolved oxygen values (optionally from a dissolved oxygen probe) collected with a frequency of e.g., less than about once every three hours. In other exemplary embodiments, the mean dissolved oxygen content is the mean of measured dissolved oxygen values (optionally from a dissolved oxygen probe) collected with a frequency of every hour, every 30 minutes, or every 15 minutes.

As used herein “a daily mean of a dissolved oxygen content” refers to the mean of measured dissolved oxygen values, wherein the values have been measured during the presence of sunlight or artificial light.

In exemplary embodiments, the stream containing algal growth medium, algal cells and/or prokaryotes is treated in the presence of sunlight. Treatment in the presence of sunlight can include performing the treating of the stream (i.e., the reducing or excluding of light exposure to the stream) during times of the day when sunlight is present.

In exemplary embodiments, the algal harvester feed stream has a mean dissolved oxygen content of less than 60%, less than 55%, less than 50%, less than 45%, less than 40%, less than 35%, less than 30%, less than 25%, less than 20%, less than 15%, less than 10%, and/or less than 5%, of the maximum mean dissolved oxygen content observed in the stream containing algal growth medium, algal cells and/or prokaryotes; optionally during a 24-hour period.

In exemplary embodiments, the stream containing algal growth medium, algal cells and/or prokaryotes has a measured daily maximum value of dissolved oxygen content of greater than about 9 mg DO/liter.

In exemplary embodiments, the method includes at least one or more of the following steps: i) adding the algal harvester feed stream to a harvester with a gaseous stream including an inert gas, optionally wherein the inert gas is selected to be at least one or more of nitrogen, a noble gas, carbon dioxide, or a combination thereof; ii) adding an antioxidant, optionally a water-soluble antioxidant, selected from, but not limited to, ascorbic acid, propionic acid, carnosic acid, uric acid, glutathione, and combinations thereof, to the algal harvester feed stream; iii) utilizing an inert flotation gas in an adsorptive bubble separation method in the harvester; and/or iv) utilizing sodium sulfite to deoxygenate with a cobalt catalyst.

In exemplary embodiments, the stream containing algal growth medium, algal cells and/or prokaryotes and/or the algal harvester feed stream contains Dunaliella sp., Dunaliella salina, and/or Dunaliella bardawil.

In exemplary embodiments, the stream containing the algal growth medium, algal cells and/or prokaryotes and/or the algal harvester feed stream contains at least about 10,000 cells of algae or microalgae, such as Dunaliella sp., Dunaliella salina, and/or Dunaliella bardawil, per milliliter. For example, at least 20,000, at least 30,000, at least 40,000, at least 50,000, at least 60,000, at least 70,000, at least 80,000, at least 90,000, or at least 1,000,000 cells of algae or microalgae are contained in the stream containing algal growth medium, algal cells and/or prokaryotes and/or the algal harvester feed stream.

In exemplary embodiments, the method includes recovering algal products from the algal harvester feed stream.

The stream containing algal growth medium, algal cells and/or prokaryotes can have a degree of salinity from about 5 wt % to about 27 wt %. In exemplary embodiments, the stream can be saturated with salt (e.g., for sodium chloride, the saturating degree of salinity is about 27 wt % at 25° C.).

The expression “salinity” refers to the total amount of dissolved salts that can be present within a biomass and/or the medium in which the biomass resides (e.g., the stream containing the algal growth medium, algal cells and/or prokaryotes, the algal harvester feed stream, any algal culture medium steam leaving the harvester and/or any algal culture medium steam leaving a biological polishing system or unit).

Salts which can be dissolved and found in the biomass and/or its medium include, but are not limited to, those found in natural waters such as sodium chloride, magnesium chloride, calcium and magnesium sulfates, bicarbonates, and carbonates. In more general terms, salinity is indicated by the water source, such as a freshwater, a brackish water, a saline water, and a brine. Ranges of salinity are associated with these general terms and these ranges are defined as <0.05 wt % for freshwater, 0.05-3 wt % for brackish water, 3-5 wt % for saline water, and >5 wt % for a brine.

As used herein, “wt %” refers to a dry mass of a component in a solution in grams divided by 100 grams of the solution.

The stream containing algal growth medium, algal cells and/or prokaryotes, the algal harvester feed stream and/or any other stream implicated in the present methods or systems can have a salinity that is about 5% or greater than 5%, about 6% or greater than 6%, about 7% or greater than 7%, at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, at least about 25 wt %, at least about 26 wt %, or at least about 27 wt %. In exemplary embodiments, at least one stream implicated in the present methods or systems is saturated with salt.

In exemplary embodiments, at least one stream implicated in the present methods or systems has a salinity that is about 5 wt % to about 27 wt % or saturation, from about 7 wt % to saturation, from about 10 wt % to saturation, from about 5 wt % to about 25 wt %, from about 20 wt % to saturation, from about 5 wt % to about 20 wt %, from about 10 wt % to about 20 wt %, from about 5 wt % to about 15 wt %, from about 10 wt % to about 15 wt %, or from about 5 wt % to about 10 wt %.

In exemplary embodiments, the salinity of the algal harvester feed stream has about the same salinity or a salinity that does not differ more than 10 wt %, more than 5 wt %, more than 4 wt %, more than 3 wt %, more than 2 wt % or more than 1 wt % between at least one of the streams disclosed herein.

In exemplary embodiments, the salinity of the stream containing algal growth medium, algal cells and/or prokaryotes is from about 5 wt % to saturation.

In exemplary embodiments, the method includes performing the treating of the stream containing algal growth medium, algal cells and/or prokaryotes at a temperature range selected from about −10° C. to about 60° C., from 0° C. to about 50° C., and/or from about 20° C. to about 45° C.

In exemplary embodiments, the method includes at least one or more of: obtaining the stream containing algal growth medium, algal cells and/or prokaryotes from an algal aquaculture pond; recovering algal products from the harvester; and/or recycling algal culture medium from the harvester to the algal aquaculture pond.

In exemplary embodiments, the method includes reducing light exposure of the stream containing algal growth medium, algal cells and/or prokaryotes by at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, and/or 99%.

In exemplary embodiments, the method includes treating the stream containing algal growth medium, algal cells and/or prokaryotes in a covered canal in the presence of external sunlight (i.e., sunlight outside of the covered canal).

Disclosed herein is also an exemplary use of the algal harvester feed stream containing a dissolved oxygen content of less than about 8 mg dissolved oxygen (DO)/liter in methods for extracting algae and/or products (e.g., algal oil, proteins and carotenoids) from a biomass.

In exemplary embodiments, the dissolved oxygen content of the algal harvester feed stream and/or the stream containing algal growth medium, algal cells and/or prokaryotes is a mean taken over a 24-hour period.

Another aspect of the present disclosure is a system for reducing degradation and/or loss of algal products by oxidation during harvesting of a biomass containing algal cells, the system including at least one or more of: a covered canal configured to reduce or eliminate light exposure of a stream containing an algal growth medium, algal cells and/or prokaryotes; and/or a harvester configured to communicate with the covered canal. The system can also include pipes, for example those made of plastic, opaque plastic, ceramic, cement, and other suitable materials.

In exemplary embodiments, the system includes at least one of the following: a bioreactor (e.g., a harvester); at least one pond (e.g., an algal aquaculture pond); a pump for feeding an algal harvester feed stream to the bioreactor (e.g., a harvester feed pump); means for recycling algal culture medium from the bioreactor to the at least one pond, the means including a pipe, tube and/or canal, and/or a second reactor (e.g., a biological polishing system or unit).

In exemplary embodiments, the system includes a covered canal configured to receive a stream containing algal growth medium, algal cells and/or prokaryotes (e.g., from an algal aquaculture pond); and/or configured to retain and/or release an algal harvester feed stream.

In exemplary embodiments, the bioreactor or harvester is configured to recover algal products from an algal harvester feed stream and discharge algal culture medium.

In exemplary embodiments, the system includes a covered canal configured to reduce light exposure to a stream containing algal growth medium, algal cells and/or prokaryotes to produce an algal harvester feed stream with a dissolved oxygen content of less than about 8 mg dissolved oxygen (DO)/liter. In exemplary embodiments, the system is configured to calculate the dissolved oxygen content in the algal harvester feed stream as a mean taken over a 24-hour period.

In exemplary embodiments, the system is at least partially a gravity flow system, wherein gravity flow is provided at least from the at least one or more ponds (e.g., algal aquaculture ponds) to a covered canal and/or from the covered canal to the at least one or more bioreactors (e.g., harvester).

In exemplary embodiments, the system includes a valve, a weir, and/or a pump to control a treating time or a residence time of a stream containing algal growth medium, algal cells and/or prokaryotes in a covered canal.

In exemplary embodiments, the bioreactor or harvester contains or has an adsorptive bubble separation unit. The adsorptive bubble separation unit can include a flotation aid, a frother, a collector, a depressor, an activator or any combination thereof.

In exemplary embodiments, the bioreactor or harvester includes more than one adsorptive bubble separation unit.

In exemplary embodiments, the system includes at least one or more of the following: an aquaculture pretreatment unit configured for conditioning an aqueous medium with at least algal nutrients to obtain an algal growth medium, the aquaculture pretreatment unit being arranged to feed the algal growth medium via a water feed line to an algal aquaculture pond, optionally by gravity feed; at least one biological polishing system or unit containing a population of microorganisms that metabolize glycerol, optionally in communication with a harvesting zone containing at least one bioreactor or harvester and/or at least one pond (e.g., at least one algal aquaculture pond), optionally by at least one or more of a pipe, a tube, and/or a canal; and/or one or more ponds for crystallizing salt, optionally in communication with the biological polishing system or unit;

Use of the systems disclosed herein for reducing degradation, optionally degradation by oxidation, and/or loss of algal cells or products thereof during harvesting of a biomass containing algal cells, is also disclosed.

Another aspect of the present disclosure is an algal harvester feed stream composition that includes algae or products thereof, algal growth medium, and has a dissolved oxygen content of less than about 8 mg dissolved oxygen (DO)/liter.

In exemplary embodiments, the composition has a dissolved oxygen content of less than about 7 mg DO/liter, 6 mg DO/liter, 5 mg DO/liter, 4 mg DO/liter, 3 mg DO/liter, 2 mg DO/liter, and/or 1 mg DO/liter.

The algal harvester feed stream compositions disclosed herein can be obtained by any one or combination of the exemplary methods disclosed herein.

Another aspect of the present disclosure is an algal biomass containing algal oil, proteins and/or carotenoids and obtained by any method of the present disclosure, wherein the algal oil, proteins and/or carotenoids are recoverable from the algal biomass. The algal biomass can be present in the algal harvester feed stream and/or the stream containing algal growth medium, algal cells and/or prokaryotes.

Surprisingly, the methods and systems disclosed herein have been found to significantly reduce the amount of dissolved oxygen in an algal harvester feed stream being fed to a harvester by subjecting a stream containing algal growth medium, algal cells and/or prokaryotes to darkness or reduced light. Without being bound to any theory, the reduced amount of dissolved oxygen occurs from both the lower salinity bacteria and halo-tolerant bacteria present in the stream containing algal growth medium, algal cells and/or prokaryotes consuming oxygen during respiration and secreting carbon dioxide.

This respiration can diminish the amount of dissolved oxygen in the stream containing algal growth medium, algal cells and/or prokaryotes in a timeframe that can produce an algal harvester feed stream that contains a dissolved oxygen content of less than about 8 mg oxygen/liter

It has also been surprisingly discovered that the methods and systems disclosed herein can reduce the amount of dissolved oxygen in a biomass containing algal cells and produce enriched and easily purified biological algal products downstream from a harvester. Significant amounts of oxygen, e.g. dissolved oxygen, can be effectively removed from the stream containing algal growth medium, algal cells and/or prokaryotes without the need for expending large amounts of energy, utilizing expensive equipment or chemicals, or generating unnecessary waste.

There are several exemplary advantages that the methods and systems of the present disclosure have over known methods. For example, the methods disclosed herein do not require the use of inert gas to reduce the oxygen content of the algal harvester feed stream, thereby saving on equipment and operating costs. Handling an inert gas also requires significant safety considerations in order to maintain high operator safety standards.

The methods and systems disclosed herein also do not require the use of an antioxidant to remove contaminants. Adding an antioxidant requires experimentation to determine the best antioxidant and its dosage to be used. This can lead to the use of expensive antioxidants and can also introduce a potential contaminant into the system where the discharge is returned to the algal aquaculture pond. The contaminants must then be removed or reduced to acceptable levels that need to be determined.

The methods and systems disclosed herein also do not require the use of an inert flotation gas to harvest an algal biomass. Using an inert flotation gas in an adsorptive bubble separation method for the algal harvesting requires both capital costs for gas storage and refilling, as well as operating costs to obtain the inert gas. There are also additional costs for safely handling the inert gas. The methods and systems disclosed herein do not require additional moving equipment, such as pumps.

The methods and systems disclosed herein do not require the addition of sulfite and cobalt catalysts. The potential addition of sulfite and cobalt can be limited by the capital costs of the additional unit and cobalt catalyst. Additionally, the sulfite and cobalt catalyst replacement can incur additional operating costs. Finally, the addition of sulfite would result in a requirement to handle sulfate, which must be considered for discharge regulations.

Regardless of the method used for controlling the oxygen level fed to the harvester, the dissolved oxygen content in the algal harvester feed stream can also impact the corrosive aspect of the brine and the material of construction of the harvester and associated equipment. Corrosion rates can increase with increasing oxygen concentration. Thus, the corrosion rate of equipment in the algal harvester and other downstream processes can be positively impacted by reducing the dissolved oxygen content as described herein.

It is also surprising that the systems of the present disclosure can be used to establish a continuous flow method or aquaculture system, thereby increasing the efficiency of the systems by saving resources and reducing environmental waste.

In exemplary embodiments, the systems disclosed herein can include chemicals that are commonly used in adsorptive bubble separation units, specifically collectors, depressors, frothing agents, and frothers. Collectors can selectively render one or more molecules or natural products in the biomass hydrophobic, thereby assisting in the method of collecting biomass on gas bubbles. Activators can aid in the adsorption of the collector to certain molecules or natural products in the biomass, thereby increasing the number of those molecules or products which become hydrophobic. Depressors can inhibit the adsorption of the collector to undesirable molecules or natural products in the biomass, thereby decreasing the number of those molecules or products which become hydrophobic. Also, frothing agents and frothers can be added to the harvester to assist in the formation of a stable froth containing hydrophobic molecules or natural products from the biomass on the surface of a liquid.

The harvester used in the methods and systems disclosed herein can include one or more units or equipment for the centrifugation, flocculation, sedimentation, and/or filtration of the biomass. Additional harvesting equipment include, but are not limited to, an adsorptive bubble separation unit, a filter, a deep bed filter, a belt press, a screw press, a centrifuge, an adsorber, a sedimentation unit, a mechanical flotation unit, a dissolved gas flotation unit, a froth flotation unit, a flocculation unit, or any combination thereof. Such equipment or units are described e.g. in U.S. Pat. No. 5,776,349.

Any sedimentation unit or equipment known in the art can be present in connection or communication with the harvester. For example, sedimentation units that can add alum to the biomass and/or not agitate the biomass when in a stream or media can be included within the harvester. Other possible sedimentation units that can be present in communication with the harvester are those that can add ferric chloride and/or polymers or ions that cause flocculation of the biomass. Cyclone sedimentation units can also be included with the harvester.

Deep bed filtration equipment can also be present in communication with the harvester. This equipment can be used to preconcentrate the biomass prior to an adsorptive bubble separation method. Deep bed filtration relies upon a bed of granular media, e.g., sand, through which a stream containing the biomass flows downward under gravity. The biomass can be deposited in the pores of the granular media and in the interstitial spaces between the grains of media. Deep bed filtration should not be confused with straining filtration. Straining takes place on the surface of a mesh or fabric and is only suitable to pre-concentrate a stream with natural products that will not blind the filtration equipment.

The harvester can also include equipment or units that can rupture cells contained in the biomass. The biomass includes cellular material that contains natural products, such as algal oil, proteins, and carotenoids. Therefore, rupturing the cell wall and/or cell membrane of the cellular material can release natural products that can be later extracted, isolated and/or purified. Cell rupturing can be achieved by a number of methods which include, but are not limited to, chemical, physical or mechanical methods. Chemical methods can include enzymatic digestion, detergent solubilization, lipid dissolution with a solvent, and alkali treatment (lipid saponification). Physical methods can include osmotic shock, decompression, sonication, heat treatment, and freeze-thawing. Mechanical methods can include grinding, high shear homogenization, passing the feedstock stream across a pressure drop, and pressure extrusion.

Other cell disruption methods which can be used include pumping the algal harvester feed stream at high pressures through a restricted orifice valve. An equipment which can perform this disruption method is, as an example, the MICROFLUIDIZER™ cell disruption equipment of Microfluidics, Newton, MA, US, which utilizes pressures of about 5,000 to 40,000 psig (345-2760 bar).

A mill, such as a vibratory mill, can also be present in connection or communication with the harvester and used to rupture cellular material in the biomass as described in U.S. Pat. No. 9,358,553.

In exemplary embodiments wherein the biomass contains algae or microalgae components (such as algal biomass), the harvester can include equipment that is capable of fracking the biomass. The partial rupturing of algae is referred to as fracking. Fracking can take place in any device known in the art in which algae or microalgae can be partially ruptured including, but not limited to, a vibratory mill, a French press, a pump, an agitated vessel, or combinations thereof. Fracked algae are preferable to completely ruptured algae due to the difference in size of the resulting particles. Particles resulting from fracking algae are larger than the particles resulting from the complete rupturing of algae and thus adsorptive bubble separation methods could be more effective when larger particles are present. Fracking the algae or microalgae can produce fracked cells possessing hydrophobic components while still retaining a significant portion of the intracellular material within the cellular membrane. This can result in increased recovery of the intracellular material and the natural products contained within the cells.

In exemplary embodiments, the methods disclosed herein include one or more of the following steps: i) adding the algal harvester feed stream with a gaseous stream including an inert gas, optionally wherein the inert gas is nitrogen, a noble gas, carbon dioxide, or a combination thereof; ii) adding an antioxidant, optionally a water-soluble antioxidant, selected from ascorbic acid, propionic acid, carnosic acid, uric acid, glutathione, and/or combinations thereof, to the algal harvester feed stream; iii) utilizing an inert flotation gas in an adsorptive bubble separation process in the harvester; and/or iv) utilizing sodium sulfite to deoxygenate the stream containing algal growth medium, algal cells and/or prokaryotes and/or the algal harvester feed stream with a cobalt catalyst. For example, the inert gas can be sparged into the stream containing algal growth medium, algal cells and/or prokaryotes and/or the algal harvester feed stream. In exemplary embodiments, the sparging occurs in a pipe or covered canal before the harvester. In other exemplary embodiments, steps i), ii), iii) and/or iv) occur in a pipe/tube/covered canal or before the harvester. In exemplary embodiments, the cobalt catalyst in step iv) is cobalt chloride. In exemplary embodiments, the cobalt catalyst is cobalt chloride at a dose of no more than 0.05 mg/L. In exemplary embodiments, the sodium sulfite is 1.5 times that stoichiometrically required by the equation Na₂SO₃+0.5 O₂=Na₂SO₄, such as to ensure complete deoxygenation. The sodium sulfite used, for example, is about 7.875 mg/L or 11/8 mg/mg O₂.

Using an algal harvester feed stream with a reduced dissolved oxygen content reduces the potential exposure of algal products to oxygen in the harvester and thus better preserves the algal products from degradation and/or loss due to oxidation.

Recycling algal culture medium discharged from the harvester can control the salinity and other components including but not limited to minerals, heavy metals, biological components, or combinations thereof, in the algal aquaculture pond. Purged algal culture medium can contain lower levels of oxygen compared to the algal harvester feed stream.

In exemplary embodiments, the systems described herein include at least one of the following: at least one pond (e.g., an aquaculture pond), a covered canal, and at least one harvester. In exemplary embodiments, the system includes at least one pond (e.g., an algal aquaculture pond), a pump configured to feed an algal harvester feed stream to the harvester; and/or a means for recycling culture medium from either the harvester to the at least one pond and/or from the at least one bioreactor to the at least one pond.

In exemplary embodiments, the covered canal is configured to receive a stream containing algal growth medium, algal cells and/or prokaryotes, optionally from an algal aquaculture pond; and/or is configured to retain and/or release an algal harvester feed stream. In exemplary embodiments, the harvester is configured to convert an algal harvester feed stream into an algal culture medium and/or algal products in an algal growth medium.

The covered canal can be configured to reduce light or induce darkness to the stream containing algal growth medium, algal cells and/or prokaryotes to produce an algal harvester feed stream with a dissolved oxygen content less than about 8, about 7, about 6, about 5, about 4, about 3, about 2, and/or about 1 mg dissolved oxygen (DO)/liter.

In exemplary embodiments, the system includes a second reactor in direct or indirect communication with the harvester. In exemplary embodiments, the second reactor is in communication with the harvester to provide a continuous algal harvesting system.

The second reactor can include or be a polishing unit (e.g., a bioreactor such as a biological polishing unit) that is either a pond open to the atmosphere, a mechanically agitated pond (e.g., raceway pond design), a pond without mechanical agitation, a pond closed to the atmosphere (e.g., a greenhouse-covered pond), a bio-film based reactor (e.g., a biofilter, a trickle bed reactor or fluidized bed bioreactor), a moving bed biofilm reactor, a closed or semi-closed bioreactor (e.g., a fermenter, a pipeline/vessel containing a UV-radiation source, a pipeline/vessel containing an oxidizing agent, a protein skimmer, a fixed-film bioreactor), or any combination thereof. In exemplary embodiments, the second reactor includes or is a biological polishing system, the polishing system being either an open pond, bioreactor, or combination thereof.

The microorganisms inhabiting the second reactor can include any one or combination of microorganisms disclosed herein.

In exemplary embodiments, the second reactor contains microorganisms that include or are heterotrophic and/or mixotrophic microorganisms that can produce useful biological materials, such as CO₂, lipids, proteins and other molecules such as macromolecules.

In exemplary embodiments, the second reactor contains microorganisms that include halophilic microorganisms that are optionally capable of growing under saturating salt conditions and/or autotrophic microorganisms.

In exemplary embodiments, the second reactor contains microorganisms that include one or more anaerobic microbes, and/or one or more aerobic microbes.

In exemplary embodiments, the microorganisms include(s) one or more bacteria and/or archaea selected from Halobacterium, Halomonas, Haloquadratum, Halosimplex, Haloferax, Haloarcula, Halorubrum, Natrialba, Natronobacterium, Natronococcus, Salinibacter, Spiribacter, and any combination thereof.

In exemplary embodiments, the microorganisms contain eukaryotic algae cells and prokaryotic cells. Such prokaryotic cells can include bacteria and/or archaea cells. The ratio of eukaryotic algae cells (cells/ml) to prokaryote cells (cells/ml) can be higher in the algal aquaculture pond than in the second reactor and/or the ratio of prokaryote cells (cells/ml) to eukaryotic algae cells (cells/ml) can be higher in the second reactor containing microorganisms than in the algal aquaculture pond.

In exemplary embodiments, the ratio of eukaryotic algae cells in the algal aquaculture pond to eukaryotic algae cells in the second reactor containing microorganisms is more than 1:1, more than 10:1, more than 100:1, more than 1000:1, more than 5000:1, or more than 10,000:1 and/or the ratio of the prokaryote cells in the algal aquaculture pond containing microorganisms to the prokaryote cells in the second reactor containing microorganisms is less than 1:1, less than 1:2, less than 1:5, less than 1:10, less than 1:50, less than 1:100, or less than 1:1000.

In exemplary embodiments, the systems described herein include a first and/or a second recovery line. The first recovery line can be in communication with the harvester and can mediate the removal of the algal products, and optionally salt, from the harvester. The first recovery line can be configured to recover microbial, e.g. algal, biomass from the harvester. The second recovery line can be in communication with the second reactor and can mediate the removal of an algal culture medium, or oxygen degradation products, or metabolites thereof, optionally including salt. A third recovery line can be included within the system and configured to recover oxygen degradation products or metabolites from the second reactor.

In exemplary embodiments, the systems described herein include a first, a second, and/or a third transport line. The first transport line can be in communication with the algal aquaculture pond and a covered canal. The first transport line can mediate the exchange of material from the algal aquaculture pond to the covered canal. The second transport line can be in communication with the covered canal and the harvester. The second transport line can mediate the exchange of material from the covered canal to the harvester. The third transport line can be configured to transport algal culture medium from the second reactor to the algal aquaculture pond.

One or more recovery lines and/or one or more transport lines can either be canals, canals lined with waterproof materials, pipes, tubes or any combinations thereof.

In exemplary embodiments, the communication between the harvester and the algal aquaculture pond and/or the communication between the harvester and second reactor is provided by a pipe, tube and/or canal.

One or more recovery lines and/or one or more transport lines can be formed from plastic, concrete, clay, and/or any material resistant to salt corrosion. In some embodiments, the transport lines are canals possessing a cover which is optionally lined with corrosion resistant materials, such as materials resistant to salt damage and/or wear.

In other exemplary embodiments, the systems described herein include a water source and a water feed line. The water feed line is in communication with a water source and the algal aquaculture pond. The water feed line can mediate the exchange of water from the water source to the algal aquaculture pond.

In exemplary embodiments, the systems described herein include a hydraulic pump. The hydraulic pump can be placed on any of the transport lines described herein and/or the water feed line.

In exemplary embodiments, a stream containing algal growth medium, algal cells, prokaryotes and biomass is fed to a harvester. The biomass is effectively separated from the stream containing an algal growth medium, algal cells and/or prokaryotes in the harvester and is removed from the harvester for optional downstream processing through a transport line in communication with the harvester.

Algal culture medium can be removed from the harvester and optionally transported to an optional second reactor along a second transport line.

In exemplary embodiments, the algal aquaculture pond is an open pond having a depth or liquid level between about 10 cm and about 500 cm. In other exemplary embodiments, the depth of the pond is between about 10 cm and about 400 cm. In other exemplary embodiments, the depth of the covered canal promoting the reduction of dissolved oxygen content is between about 0.2 to about 10 meters.

In exemplary embodiments, the systems or methods described herein are operated under continuous flow conditions optionally through all transport pipes and through the reactors and harvester. The systems and/or methods described herein can also be operated under non-continuous flow conditions e.g. through one or more of the transport pipes, with lines available to remove the algal products at each step.

In exemplary embodiments, the systems described herein are at least partially gravity flow systems, wherein gravity flow of the material is provided from at least one pond (e.g., an algal aquaculture) to the covered canal, from the covered canal to the harvester, and optionally from the harvester to a second reactor and/or at least one biological polishing system, and/or from the second reactor and/or at least one biological polishing system to the at least one pond. In exemplary embodiments, material from the algal aquaculture pond to the covered canal, material from the covered canal to the harvester, and optionally the material from the harvester to the second reactor, at least one biological polishing system and/or at least one pond is/are transported through transport lines by gravity flow.

In addition to describing systems for efficient deoxygenation, e.g. removal or reduction of dissolved oxygen, another aspect of the present disclosure relates to methods using the steps or systems described herein to deoxygenate a stream.

### Definitions

The expression “stream” or “aqueous stream” as used herein, refers to a dispersion containing water and optionally algal biomass, at least one salt, or any combination thereof. The salt can be any one or combination of salts found in sea water, terminal lakes, or aquifers. For example, the stream is a stream containing an algal growth medium, algal cells and/or prokaryotes. In another example, the stream is an algal harvester feed stream.

The streams disclosed herein can also include combinations of ions found in seawater.

The streams disclosed herein can contain concentrations of salts which range from trace amounts to saturating amounts. Suitable terms to describe the salinity or salt concentration of the stream range from fresh water, brackish water, salt water, brine, and saturated brine, respectively, as the salt concentration in the stream increases. The desired concentration of salt in the stream can depend on the type of microorganisms.

Various combinations of ions found in seawater can be included in any of the streams disclosed herein. Suitable ion combinations can be derived from one or more of the following sources including: water derived from streams as known to one skilled in the art, lakes, rivers, or other sources associated with fresh water; water derived from underground aquifers that can include various ion concentrations; water derived from industrial, agricultural, or municipal sources that have or have not received treatment; or water derived from brackish sources where fresh water is combined with sea water or ocean water in various proportions; sea water or ocean water that can be derived from the various seas and oceans located around the globe; water derived from terminal lakes; or combinations thereof.

The combination of ions can be derived directly from these sources or can be derived by evaporating the desired amount of water from any of these sources, thereby providing a desired ion-rich solution for use in the algal aquaculture pond and/or any stream disclosed herein. An example of an ion combination source is disclosed e.g. in U.S. Pat. No. 6,986,323. Other examples include the evaporation of ancient sea waters that form terminal lakes, such as the Great Salt Lake in Utah, and that form various aquifers. The combination of ions can result up to and include crystallizers wherein sodium chloride ions are precipitated.

The streams disclosed herein can contain a water content of about 99.9 wt % to about 95 wt %, about 95 wt % to about 90 wt %, about 90 wt % to about 85 wt %, about 85 wt % to about 80 wt %, about 80 wt % to about 70 wt %, about 70 wt % to about 60 wt %, about 60 wt % to about 50 wt %, or about any range within 99.9 wt % to 50 wt % of the total weight of the stream. In exemplary embodiments, the streams disclosed herein can contain a water content greater than 50 wt % or about any range within 50 wt % to 99.9 wt %.

The stream containing algal growth medium, algal cells and/or prokaryotes, the algal harvester feed stream and/or any other stream implicated in the methods or systems disclosed herein can have any salinity mentioned in the present disclosure for said stream containing a microbial biomass, such as a salinity from about 5 wt % to about 27 wt %, from about 5 wt % to about 25 wt %, from 10 wt % to about 27 wt %, from 15 wt % to about 27 wt %, from 20 wt % to about 27 wt %, from 25 wt % to about 27 wt % or any salinity falling within the above ranges.

An algal enriched stream containing algal products and isolated from the harvester can possess a ratio of eukaryotic algae cells to prokaryote cells that is less than 1:1, less than 1:2, less than 1:5, less than 1:10, less than 1:50, or less than 1:100, or less than 1:1000.

The “biomass” can be present in any of the streams disclosed herein and can include or be a plant biomass, a microbial biomass, an algal biomass, or any combination thereof.

All of the possible plant and/or microbe species which can be included in the microorganisms can also be included within the biomass in said stream.

The biomass can also include or contain some or all of the natural products produced by the microorganisms.

The biomass content in the stream containing algal growth medium, algal cells and/or prokaryotes, the algal harvester feed stream and/or any other stream implicated in the methods and/or systems disclosed herein can be as low as about 0.05 wt %, greater than about 0.5 wt %, or greater than 1 wt %. In exemplary embodiments, the maximum biomass content in any of the streams disclosed herein is less than about 20 wt %. Any content greater than 20 wt % can slow the flow of the stream through the systems described herein, thereby leading to longer purification times and an increased chance in a blockage forming within the systems. In exemplary embodiments, the maximum content of biomass in any one of the streams disclosed herein is less than about 10 wt %, less than about 5 wt %, about 2 wt %, and/or about 1 wt %.

The biomass can include or be a conditioned biomass. As used herein “a conditioned biomass” refers to a biomass that has been treated with one or more conditioning methods before separating from the stream in the harvester. Suitable conditioning methods can include, but are not limited to, fracking methods previously disclosed or combinations thereof. Suitable harvesting methods can include, but are not limited to, adsorptive bubble separation, filtration, deep bed filtration, belt pressing, screw pressing, centrifugation, adsorption, sedimentation, mechanical floatation, froth flotation, flocculation and combinations thereof.

The biomass can include useful or valuable components. These components can be produced intracellularly from microbial cells within the biomass. These microbial cells containing the valuable components can be recovered from the algal aquaculture pond and transferred to the harvester.

The microbial biomass containing the useful or valuable components can be recovered and valorized (i.e., without separating the valuable components from the biomass) from the algal aquaculture pond and/or harvester.

In exemplary embodiments, the inputting of the stream containing algal growth medium, algal cells and/or prokaryotes, also containing the biomass, to the harvester can include feeding the stream through a hydraulic pump located on a first transport line.

Separating the biomass in the harvester can include subjecting the algal harvester feed stream including the biomass to conditioning methods that can include fracking, and harvesting that includes, but is not limited to adsorptive bubble separation, filtration, deep bed filtration, belt pressing, screw pressing, centrifugation, adsorption, sedimentation, mechanical flotation, froth flotation, flocculation and combinations thereof.

In exemplary embodiments, the separating of the biomass in the harvester includes rupturing the biomass.

Once the biomass has been effectively separated from the algal harvester feed stream and its byproducts, the biomass can be effectively removed from the harvester by recovering the biomass through a first recovery line in communication with the harvester. In exemplary embodiments, the first recovery line is formed from a material of construction that can withstand the corrosive nature of the salt. Suitable materials of construction for the first recovery line include, but are not limited to stainless steel, hastelloy, polyvinyl chloride, high-density polyethylene, glass, fiberglass reinforced plastic, and combinations thereof.

In exemplary embodiments, the methods described herein include sending purged algal culture medium to a solar evaporation salt crystallizer pond wherein the media of the purged culture medium is evaporated to produce a salt product.

In exemplary embodiments, the methods described herein include injecting an effluent stream into the algal aquaculture pond from a polishing system or second reactor to provide a continuous method. In exemplary embodiments, the methods include recovering the degradation products from the algal harvester feed stream and providing an effluent stream that is injected into the algal aquaculture pond, thereby providing a continuous reactor system.

In exemplary embodiments, the system is a continuous flow reactor system.

A “continuous flow method or reactor system” refers to a method or system that can operate at least in a semi-continuous flow and/or operation. That is, a method or system which has different streams flowing in and out of the various system components continuously.

In exemplary embodiments, the methods described herein include recycling algal culture medium into the algal aquaculture pond by flowing the algal culture medium through a transport line in communication with a biological polishing system and the algal aquaculture pond. The transport line can be in either direct or indirect communication with the biological polishing system and the algal aquaculture pond.

In exemplary embodiments, the methods described herein are performed in batch-wise operations.

The algal harvester feed stream, the stream containing algal growth medium, algal cells and/or prokaryotes and/or the stream isolated from the harvester containing algal products can contain at least one hydrophobic natural product. Possible hydrophobic natural products can include, but are not limited to, one or more lipids, algal lipids, carotenoids, fatty acids, algal fatty acids, triacylglycerols, diacylglycerols, monoacylglycerols, oils, algal oils and combinations thereof.

The carotenoids can include beta-carotene, alpha-carotene, lutein, zeaxanthin, beta-cryptoxanthin, astaxanthin, phytoene, phytofluene, lycopene, and/or combinations thereof.

Any of a variety of products can be made from the biomass including, but not limited to, biofuels, nutraceuticals, cosmaceuticals, wastewater treatment methods, spa products, animal feeds, human food, soil builders, chemical intermediates, specialty lipids, solar salt, and combinations thereof.

Biofuels that can be produced from high temperature processing of the biomass include, but are not limited to, biodiesel, green diesel, renewable diesel, methane, hydrogen, alcohols, and dried algal biomass. Algal biodiesel is produced via any transesterification method known in the art, including those which utilize two immiscible liquid phases, and those that utilize a solid acid catalyst. Green diesel can be produced by hydrogenation, cracking, or a combination thereof of the algal oil or any derivative thereof in order to produce hydrocarbons that can be used directly in the existing diesel distribution system. Methane and/or hydrogen can be produced from the biomass by any anaerobic method known in the art. Fermentation of the biomass by any method known in the art can be used to produce methanol, ethanol, butanol, n-butanol, i-butanol, other alcohols, and combinations thereof. The biomass can be torrified for the production of a soil builder or for use in combination with coal for power or steam generation. The biomass can be dried and then gasified or combusted either by itself or in combination with coal or biomass.

The biomass can be extracted to recover lipids that can be used as an animal feed ingredient, renewable plastics, renewable polymers, renewable chemicals, nutraceutical, cosmaceutical, soap or components of a soap or detergent composition, and cosmetic ingredients, including, but not limited to carotenoids, omega fatty acids, and other lipids. To produce solar salt, the biomass can be removed from solar salt works to improve the salt quality. The quality of sodium chloride, sodium carbonate, and other salts can be improved by this method. Biomass stabilized with a high temperature treatment method can also be used in animal nutrition, especially for shrimp and fish aquaculture diets. The biomass can also be treated with a high temperature method to stabilize them against degradation during transportation. Alternatively, high temperature processing could be used to stabilize the biomass prior to their storage for carbon sequestration purposes. The biomass can be used to derive valuable chemical intermediates such as fatty acids to produce polyurethanes.

Suitable animal feeds include, but are not limited to, feeds for shrimp, fish, shellfish, brine shrimp, chickens, poultry, cows, ducks, dogs, pigs, sheep, goats, and combinations thereof. The animal feeds can require the biomass to be dried, but in some cases, for example for use in shrimp and fish aquaculture diets, complete drying is not necessary as long as stabilization is sufficient.

Suitable dietary supplements include, but are not limited to alpha carotene, betacarotene, lutein, zeaxanthin, cryptoxanthin, phytoene, phytofluene, and the various cis- and trans-isomers and the various alpha, beta, gamma, delta isomers of the various carotenoids, and combinations thereof.

Suitable methods of carbon storage include, but are not limited to, burying the biomass, sinking them, hydrothermal carbonization, pyrolysis to make biochar, torrefaction and using them as a soil builder or in soil amendment, and/or combinations thereof.

Suitable methods for water and wastewater treatment include, but are not limited to, removal of BOD (biological oxygen demand), and or TOC (total organic carbon) from a water stream. This can be useful for municipal and industrial wastewater treatment methods, shrimp or fish wastewater treatment, and it can be important for the treatment of brines being used for the production of sodium chloride salt and other salts via evaporation.

Suitable methods to convert the biomass into useful compounds include, but are not limited to, torification, gasification, liquefaction, fermentation, drying, combustion, burial, and combinations thereof. Suitable applications of the torrefied biomass include, but are not limited to, a soil builder and a material to be combined with coal, wood, or other combustible material for power generation, and biochar.

Suitable applications of gasified biomass includes, but are not limited to, the production of the entire suite of products that can be produced via syngas chemistry, as described by the Gasification Technologies Council.

Suitable products from syngas include, but are not limited to, chemicals, fertilizers, power generation, substitute natural gas, hydrogen, and transportation fuels.

Suitable chemicals include, but are not limited to, hydrogen, carbon monoxide, methanol, dimethyl ether, acetic acid, propionic acid, butyric acid, acetic anhydride, methyl acetate, ethylene, propylene, olefins, and combinations thereof. Suitable fertilizers that can be produced from the syngas include, but are not limited to ammonia, ammonium nitrate, urea, and others known in the art. Suitable substitute natural gas can be generated from the syngas produced by gasifying the biomass, and this includes methane.

Suitable liquid fuels include gasoline, diesel fuel, jet fuels, and combinations thereof. All of the chemicals that are produced by Eastman Chemicals and by Sasol via their gasification methods can also be produced by the gasification of the biomass. Products produced by the utilization of syngas can also be produced by gasification of the biomass. Illustrative methods are described in U.S. Pat. No. 6,310,260, which include, for example, hydroformylation, hydroacylation (intramolecular and intermolecular), hydrocyanation, hydroamidation, hydroesterification, aminolysis, alcoholysis, hydrocarbonylation, reductive hydroformylation, hydrogenation, olefin oligomerization, hydroxycarbonylation, carbonylation, olefin isomerization, transfer hydrogenation and the like. Other methods involve the reaction of organic compounds with carbon monoxide, or with carbon monoxide and a third reactant, e.g., hydrogen, or with hydrogen cyanide, in the presence of a catalytic amount of a metal-organophosphorus ligand complex catalyst. More advantageous methods include hydroformylation, hydrocyanation, hydrocarbonylation, hydroxycarbonylation and carbonylation.

### EXAMPLES

### Example 1

A covered canal that was eight feet wide carried an algal culture of Dunaliella for 335.28 meters (1100 feet) from the algal aquaculture pond to the harvester feed pump. The mean depth of the covered canal was 0.4572 meters (1.5 feet), thus the total volume of algal culture in the canal was 373.78 cubic meters (13,200 ft3 or 98,736 gallons). The mean residence time of the algal culture in the covered canal was 14 hours. The algal culture contained 1,000,000 cells per milliliter of Dunaliella. The dissolved oxygen (DO) content in the algal culture entering the covered canal was monitored with an optical density dissolved oxygen probe supplied by YSI-Xylem symbolized in FIG. 1 as (DO-in). The amount of dissolved oxygen in the algal culture and harvester feed ranged from 31 to 0 mg DO/liter. The reduction in the amount of dissolved oxygen in the algal harvester feed stream was tracked by three measures. First, the mean residence time of the algal culture in the covered canal was specifically chosen to allow the dissolved oxygen content in the algal harvester feed stream as measured by DO-out to be reduced by more than 50% from the maximum dissolved oxygen content observed in the algal aquaculture pond during the previous 24-hour period by DO-in. This allowed the dissolved oxygen concentration in the algal harvester feed stream for this example to be reduced from an initial value of 13.5 mg DO/liter as measured by DO-in to a value of 6.95 mg DO/liter as measured by DO-out (see FIG. 1) resulting in a 51% drop in the dissolved oxygen content. This reduction in the amount of dissolved oxygen fed to the algal harvester preserved the algal products that exited the algal harvester. Second, the mean residence time of the algal culture in the covered canal allowed the dissolved oxygen concentration of the algal harvester feed to be controlled at a value of less than about 8 mg DO/liter throughout a 24-hour period as measured at DO-out. In this example, the controlled value for DO content feeding the harvester was 6.95 mg DO/liter as measured by DO-out. This reduced amount of dissolved oxygen in the algal harvester feed stream resulted in less oxidized algal products. Third, the mean residence time of the algal culture in the covered canal was specifically chosen to drive the dissolved oxygen content of algal harvester feed stream exiting the covered canal to a setpoint value of 6.95 mg DO/liter based on a dissolved oxygen reduction rate of 0.49 mg DO/liter/hour. Based on multiple studies, the rate of dissolved oxygen reduction ranges from about 0.49 and 1.7 mg DO/liter/hour. In this example, the mean residence time of the algal culture in the covered canal was controlled to be 14 hours in order to achieve the desired dissolved oxygen content in the algal harvester feed stream of 6.95 mg DO/liter based on a dissolved oxygen reduction rate of 0.45 mg DO/liter/hour.

The advantage of feeding a consistently reduced composition of dissolved oxygen to the algal harvester is the reduced oxidation of algal product, specifically the algal oil and algal carotenoids present in the algal biomass.

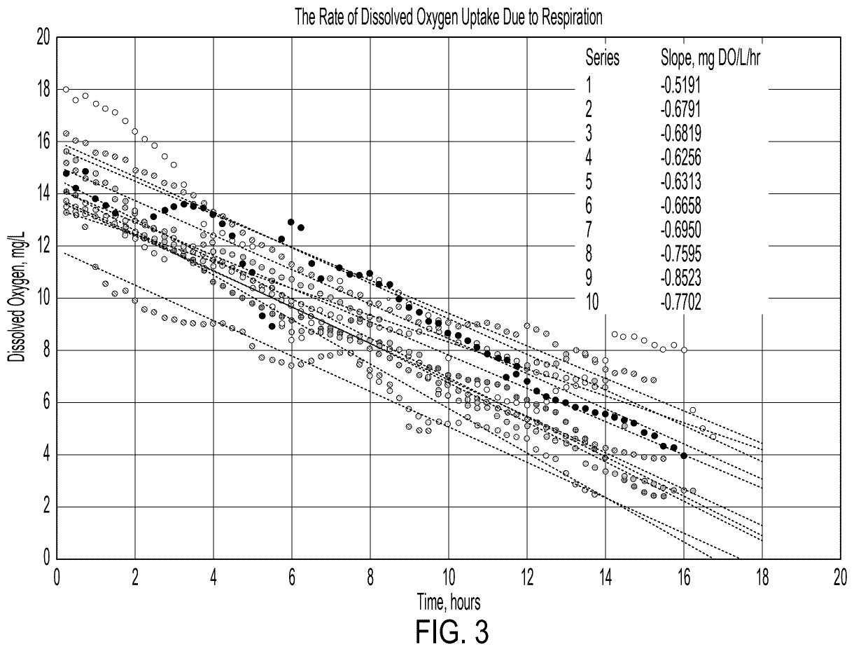

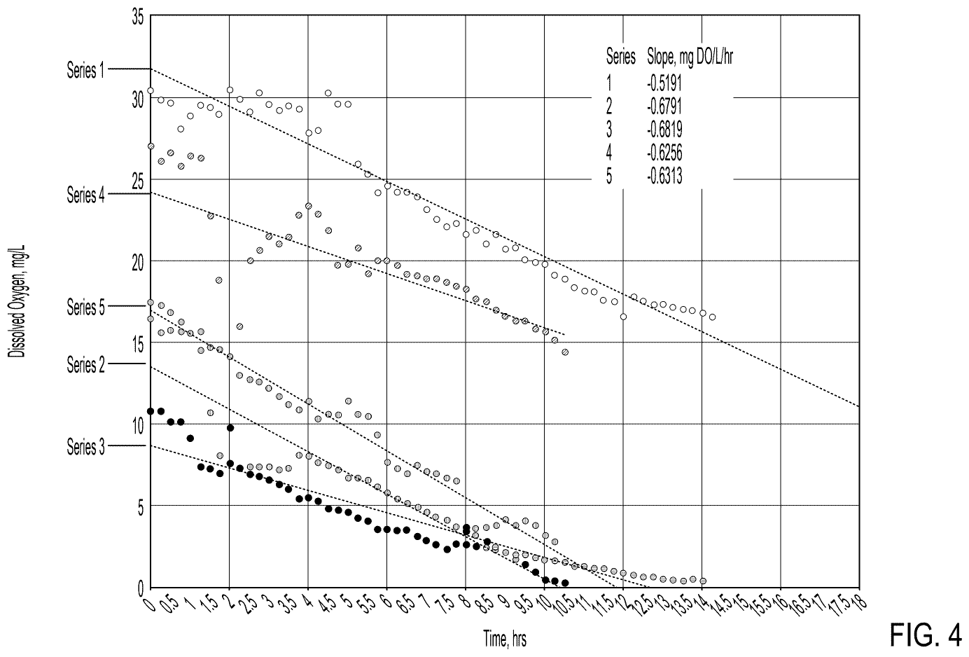

Similar data for Examples 2 through 15 are provided in Table 1 for: 1) the example number, 2) the dissolved oxygen content in the algal culture as it enters the dark region as measured by DO-in, 3) the dissolved oxygen content in the algal culture exiting the dark region as measured by DO-out, 4) the reduction percentage in DO content between DO-in and DO-out, 5) the mean residence time in the dark region, and 6) the dissolved oxygen drop rate from max to min in mg DO/liter/hour. The drop in the dissolved oxygen content in the algal culture is shown in FIG. 3 and FIG. 4.

TABLE 1
Summary of the experimental data collected for placing a culture
of Dunaliella salina in the dark as a function of time.

| | | DO in algal | | | |
|---|---|---|---|---|---|
| | DO in algal | harvester | Reduction in | Mean residence | |
| | culture | feed stream | DO content | time in | DO Drop |
| | measured by | measured by | between | covered canal | Rate from |
| Example | DO-in (mg | DO-out (mg | DO-in and | Max and Min | Min/Max, (mg |
| number # | DO/L) (Max) | DO/L)(Min) | DO-out (%) | (hours) | DO/L/hr) |
| 1 | 13.5 | 6.59 | 51 | 14 | −0.49 |
| 2 | 14.05 | 4.93 | 65 | 9.5 | −0.96 |
| 3 | 18.02 | 4.73 | 74 | 16.75 | −0.79 |
| 4 | 16.33 | 6.89 | 58 | 15.25 | −0.62 |
| 5 | 15.65 | 5.13 | 67 | 14.5 | −0.73 |
| 6 | 14.8 | 3.95 | 73 | 16 | −0.68 |
| 7 | 14.09 | 2.41 | 83 | 15.5 | −0.75 |
| 8 | 15.18 | 3.86 | 75 | 15.5 | −0.73 |
| 9 | 13.73 | 2.48 | 82 | 13.75 | −0.82 |
| 10 | 13.33 | 2.61 | 80 | 16.25 | −0.66 |
| 11 | 30.35 | 16.57 | 45 | 14 | −0.98 |
| 12 | 16.43 | 0.34 | 98 | 9.5 | −1.69 |
| 13 | 10.81 | 0.4 | 96 | 16.75 | −0.62 |
| 14 | 26.98 | 14.36 | 47 | 15.25 | −0.83 |
| 15 | 17.46 | 2.8 | 84 | 14.5 | −1.01 |

It will be appreciated by those skilled in the art that the present disclosure can be embodied in other specific forms without departing from the spirit or essential characteristics thereof. It will also be appreciated by those skilled in the art that the reduction in the dissolved oxygen level can also be achieved by reducing the mean residence time in the covered canal. For example, if only a slight reduction in the dissolved oxygen level was needed, then a shorter residence time in the covered canal would be needed and the desired residence time can be estimated by using the reduction rate of dissolved oxygen content that is measured and was found to be from about 0.49 and 1.7 mg DO/liter/hour. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

1-41. (canceled)

42. A method for harvesting a biomass containing algal cells, the method comprising:
obtaining a stream containing an algal growth medium, algal cells and/or prokaryotes from an algal aquaculture;
treating the stream by reducing or excluding the stream's exposure to light to obtain an algal harvester feed stream having a dissolved oxygen content less than about 8 mg dissolved oxygen (DO)/liter; and
feeding at least a part of the algal harvester feed stream into a harvester.

43. The method according to claim 42, comprising:
using a pipe, a tube and/or a cover in the treating of the stream.

44. The method according to claim 42, wherein the method is a continuous flow process.

45. The method according to claim 42, the treating comprising:
reducing or excluding the steam's exposure to light by flowing the stream through an opaque pipe, an opaque tube, in a covered canal, and/or a combination thereof.

46. The method according to claim 42, wherein the stream and/or the algal harvester feed stream contains bacteria and/or microalgae, and optionally has a balanced bacteria: microalgae ratio.

47. The method according to claim 42, comprising:
diminishing an amount of dissolved oxygen in the stream by respiration during the treating; and or
reducing degradation and/or loss of algae and/or products thereof in the harvester, and optionally reducing oxidation degradation of the algal harvester feed stream in the harvester.

48. The method according to claim 42, the treating comprising:
reducing or excluding the stream's exposure to light, optionally in a pipe or a tube or in a covered canal, for a treating time or a mean residence time of about 2 to about 30 hours, about 5 to about 25 hours, and/or about 8 to about 20 hours; and
controlling the dissolved oxygen content of the algal harvester feed stream by varying the treating time or the mean residence time of the stream in the covered canal or pipe or tube.

49. The method according to claim 42, comprising:
consistently reducing a dissolved oxygen content in the stream at a rate of between about 0.4 and 1.7 mg DO/liter/hour; and
wherein the dissolved oxygen content in the algal harvester feed stream is less than about 7 mg DO/liter, less than about 5 mg DO/liter, less than about 3 mg DO/liter, or less than about 1 mg DO/liter.

50. The method according to claim 42, comprising:
monitoring dissolved oxygen (DO) content of the stream and/or algal harvester feed stream using an optical density dissolved oxygen probe, and
optionally monitoring dissolved oxygen (DO) content of the stream when the stream enters a pipe, tube or covered canal and/or when the algal harvester feed stream exits the pipe, the tube or the covered canal.

51. The method according to claim 42, wherein the algal harvester feed stream has a mean dissolved oxygen content of less than 60%, less than 55%, less than 50%, less than 45%, less than 40%, less than 35%, less than 30%, less than 25%, less than 20%, less than 15%, less than 10%, and/or less than 5%, of a maximum mean dissolved oxygen content observed in the stream; and
optionally wherein the mean dissolved oxygen content of the algal harvester feed stream occurs in a 24-hour period; and/or
wherein the stream has a measured daily maximum value of dissolved oxygen concentration greater than about 9 mg DO/liter.

52. The method according to claim 42, comprising at least one or more of the following steps:
i) adding the algal harvester feed stream to the harvester with a gaseous stream including an inert gas, optionally wherein the inert gas is selected to be at least one or more of nitrogen, a noble gas, carbon dioxide, or a combination thereof;
ii) adding at least one antioxidant, optionally at least one water-soluble antioxidant, selected from the group consisting of: ascorbic acid, propionic acid, carnosic acid, uric acid, glutathione, and combinations thereof, to the algal harvester feed stream;
iii) utilizing an inert flotation gas in an adsorptive bubble separation method in the harvester; and/or
iv) utilizing sodium sulfite to deoxygenate the stream and/or the algal harvester feed stream with a cobalt catalyst.

53. The method according to claim 42, wherein the stream and/or the algal harvester feed stream contains at least about 10,000 cells of algae or microalgae per milliliter, and the algae or microalgae include at least one of Dunaliella sp., Dunaliella salina, or Dunaliella bardawil.

54. The method according to claim 42, comprising:
recovering algal products from the algal harvester feed stream.

55. The method according to claim 42, wherein the algal growth medium in the stream has a salinity of about 5 wt % to saturation.

56. The method according to claim 42, comprising:
performing the treating at a temperature range selected from about-10° C. to about 60° C., from 0° C. to about 50° C., and/or from about 20° C. to about 45° C.

57. The method according to claim 42, comprising at least one or more of:
obtaining the stream from an algal aquaculture pond;
recovering products and/or algal culture medium from the harvester; and/or
recycling algal culture medium from the harvester to the algal aquaculture pond.

58. The method according to claim 42, comprising:
reducing the stream's exposure to light by 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, and/or 99%.

59. The method according to claim 42, comprising:
treating the stream in the presence of sunlight; and/or
wherein the dissolved oxygen content of the stream and/or the algal harvester feed stream is a mean taken over 24 hours.

60. A system for reducing degradation and/or loss of algal products by oxidation during harvesting of a biomass containing algal cells, the system comprising:
a covered canal configured to reduce or eliminate light exposure of a stream containing an algal growth medium, algal cells and/or prokaryotes; and/or
a harvester configured to communicate with the covered canal.

61. The system according to claim 60, comprising at least one of the following:
at least one biological polishing system containing a population of microorganisms that metabolize glycerol, the biological polishing system being in communication with the harvester;
at least one algal aquaculture pond in communication with the covered canal;
a pump configured to feed an algal harvester feed stream to the harvester; and/or
a means for recycling culture medium from either the harvester to the at least one algal aquaculture pond and/or from the at least one bioreactor to the at least one algal aquaculture pond.

62. The system according to claim 60, wherein the covered canal is configured to receive a stream containing an algal growth medium, algal cells and/or prokaryotes; optionally from an algal aquaculture pond, and/or is configured to retain and/or release an algal harvester feed stream; and/or
wherein the harvester is configured to recover algal products from an algal harvester feed stream, and discharge algal culture medium; and/or
wherein the covered canal is configured to produce a dissolved oxygen content in an algal harvester feed stream of less than about 8 mg dissolved oxygen (DO)/liter; optionally over a 24-hour period.

63. The system according to claim 60, wherein the system is at least partially a gravity flow system, wherein gravity flow is provided from the harvester to the at least one algal aquaculture pond, from the at least one biological polishing system to the at least one algal aquaculture pond, and/or from the at least one algal aquaculture pond to the covered canal.

64. The system according to claim 60, comprising:
a weir, or a pump, to control a treating time or a residence time of an algal harvester feed stream in the covered canal; and
wherein the harvester comprises:
at least one or more of an adsorptive bubble separation unit.

65. The system according to claim 68, comprising at least one or more of the following:
an aquaculture pretreatment unit configured for conditioning a medium with at least algal nutrients to obtain algal growth medium, wherein the aquaculture pretreatment unit is arranged to feed algal growth medium to the at least one algal aquaculture pond, optionally by gravity feed;
one or more ponds for crystallizing salt, optionally in communication with the at least one bioreactor; and/or
optionally one or more pipes, tubes or canals.

66. An algal harvester feed stream composition comprising algae or products thereof and an algal growth medium, the composition having a dissolved oxygen content of less than about 8 mg dissolved oxygen (DO)/liter.

67. The composition according to claim 66, wherein the dissolved oxygen content is less than about 7 mg DO/liter, 6 mg DO/liter, 5 mg DO/liter, 4 mg DO/liter, 3 mg DO/liter, 2 mg DO/liter, and/or 1 mg DO/liter.

68. An algal biomass obtained by the method according to claim 42, wherein the algal biomass contains algal oil, proteins and/or carotenoids.
